(19) United States
(12) Patent Application Publication
(10) Pub. No.: US 2012/0022562 A1
(43) Pub. Date: Jan. 26, 2012

(54) DEVICE TO DETECT INTERNAL BLEEDING

(75) Inventor: Martin Willard, Burnsville, MN (US)

(73) Assignee: BOSTON SCIENTIFIC SCIMED, INC., Maple Grove, MN (US)

(21) Appl. No.: 13/188,657

(22) Filed: Jul. 22, 2011

Related U.S. Application Data
(60) Provisional application No. 61/367,318, filed on Jul. 23, 2010.

(51) Int. Cl. A61B 17/08; A61B 5/05

(52) U.S. Cl. 606/151; 600/407

(57) ABSTRACT

A vascular closure device, comprising an anchor member; a compressible plug; a locking mechanism; and a sensor probe, the sensor probe including a first electrically conductive member having a proximal end and a distal end and a second electrically conductive member having a proximal end and a distal end, the first electrically conductive member being electrically insulated from the second electrically conductive member from the proximal end to the distal end.

This application claims priority to U.S. Provisional Application No. 61/367,318 filed Jul. 23, 2010.

### FIELD

The present invention relates to devices to detect internal bleeding and methods for using the devices to detect internal bleeding.

### BACKGROUND

In many medical procedures, such as, for example, balloon angioplasty and the like, an opening can be created in a blood vessel or arteriotomy to allow for the insertion of various medical devices which can be navigated through the blood vessel to the site to be treated. For example, a guidewire may first be inserted through the tissue tract created between the skin, or the epidermis, of the patient down through the subcutaneous tissue and into the opening formed in the blood vessel. The guidewire is then navigated through the blood vessel to the site of the occlusion or other treatment site. Once the guidewire is in place, an introducer sheath can be slid over the guide wire to form a wider, more easily accessible tract between the epidermis and the opening into the blood vessel. The appropriate medical device can then be introduced over the guidewire through the introducer sheath and then up the blood vessel to the site of the occlusion or other treatment site. Such a treatment is generally known as a percutaneous coronary intervention (PCI).

Once the procedure is completed, the medical devices or other equipment introduced into the vessel can be retracted through the blood vessel, out the opening in the blood vessel wall, and out through the tissue tract to be removed from the body. The physician or other medical technician is then presented with the challenge of trying to close the opening in the blood vessel and/or the tissue tract formed in the epidermis and subcutaneous tissue. One option for closing the opening in the blood vessel is to apply manual pressure to the wound opening. Another option is to use a vascular closure device to mechanically close and seal the opening in the blood vessel.

However, even when an appropriate technique and/or device is used to close the blood vessel following the PCI, serious complications may ensue. Among the most serious of complications is retroperitoneal hemorrhage (RPH) and hematoma, where blood collects internally around the entry site into the blood vessel. The retroperitoneum can collect a large amount of blood with few and ambiguous symptoms until hypervolemia occurs. Any delays in diagnosing RPH can significantly increase morbidity and potentially fatal complications. Retroperitoneal hemorrhage is thought to occur in approximately 0.5 percent of all procedures and may have an associated mortality rate of between about 4 percent and 10 percent. Early diagnosis of RPH or potential RPH is associated with better clinical outcomes. The most common symptoms are abdominal pain, groin pain, back pain, diaphoresis (perspiration), bradycardia (slow heartbeat) and hypotension. Patients may only report one of these symptoms, none of which are strongly indicative of RPH or even particularly unusual following a major interventional procedure. There is thus a strong and ongoing need for devices and procedures to detect RPH and other internal bleeding.

### BRIEF SUMMARY

The following summary is provided to facilitate an understanding of some of the innovative features unique to the present disclosure and is not intended to be a full description. A full appreciation of the disclosure can be gained by reference to the entire application.

One aspect pertains to a method of using a sensor probe that is inserted into the entry wound and placed next to the punctured blood vessel or other area to be monitored. The sensor probe may be introduced during the vessel closure procedure or after, but in any case prior to the closure of the entry wound in the skin. The sensor probe can be connected to a monitoring unit, which can provide a signal when blood is detected.

Another aspect of the disclosure pertains to the sensor probe. The sensor probe may utilize resistive sensing technology, where the resistance between two spaced-apart conductors is monitored and where the resistance decreases in the presence of blood. The sensor probe may also utilize other sensing technologies such as capacitive or optical. In another aspect, there may be a non-conductive coating that dissolves in the presence of blood, where the dissolution of the coating allows the presence of the blood to complete a circuit.

### BRIEF DESCRIPTION OF THE DRAWINGS

Embodiments of the present disclosure may be more completely understood in consideration of the following detailed description in connection with the accompanying drawings, in which:

FIG. 1 is a schematic diagram of a device for sealing a vessel puncture incorporating a sensor probe according to an embodiment of the present disclosure;

FIG. 2 is a schematic diagram of the device of FIG. 1 in an installation configuration;

FIG. 3 is a schematic diagram of a device for sealing a vessel puncture incorporating a sensor probe according to an embodiment of the present disclosure;

FIG. 4 is a schematic diagram of a sensor probe inserted into a wound proximate a blood vessel;

FIG. 5 is a schematic diagram of the distal end of a sensor probe;

FIG. 6 is a cross-sectional view of the sensor probe of FIG. 5;

FIG. 7 is a schematic diagram of the distal end of a sensor probe;

FIG. 8 is a schematic diagram of the distal end of a sensor probe;

FIG. 9 is a cross-sectional diagram of the sensor probe of FIG. 8; and

FIG. 10 is a schematic diagram of a device for sealing a vessel puncture incorporating a sensor probe according to an embodiment of the present disclosure.

While the embodiments of the present disclosure are amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

The sensor probe is generally a flexible elongate member. The sensor probe may be incorporated into various medical devices and procedures in a wide variety of ways, some of which are described below. In general, the sensor probe may be substituted in some instances for a flexible elongate member of a medical device, thereby incorporating the sensing ability into the medical device. In other embodiments, the sensor probe may be attached to a medical device as an additional component of the medical device. In still other embodiments, the sensor probe may be a separate medical device that is introduced separately from other medical devices.

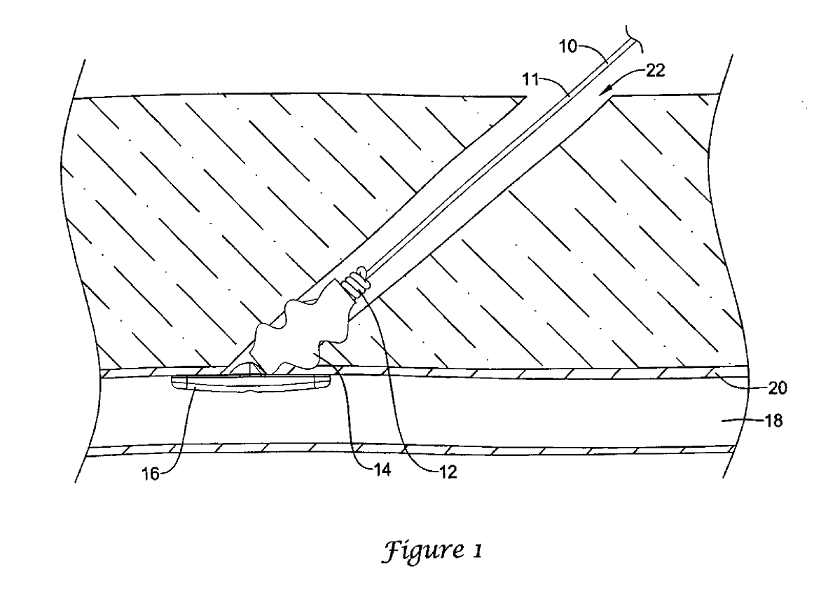

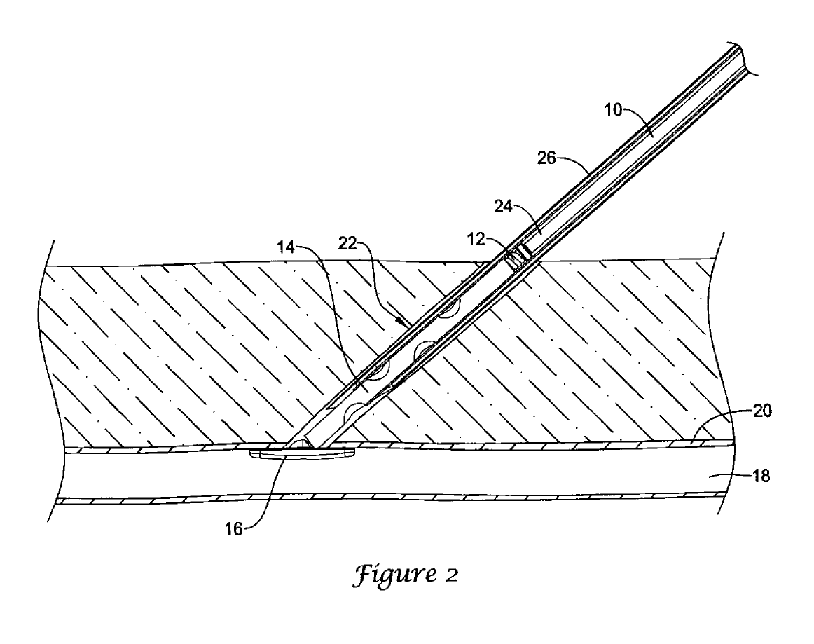

For example, FIG. 1 is a schematic diagram of a vascular closure device 10 for sealing a vessel puncture incorporating a sensor probe according to an embodiment of the present disclosure. This particular device 10 includes a plug 14, a locking element 12 and an anchor 16. A general overview of the use of such a device may be understood with reference to both FIGS. 1 and 2. In FIG. 2, it can be seen that the plug 14 is introduced through a wound 22 in a guide sheath 26. A pusher tube 24 is positioned above the locking element 12. Once the anchor 16 is properly positioned, the device 10 is held in place by suture 11 while the pusher tube 24 is advanced distally to compress the plug 14 longitudinally and expand it laterally as shown in FIG. 1 to seal the lumen 20 of the blood vessel 18. The suture 11 is an elongate flexible element, and in this embodiment the suture 11 can be a sensor probe. In conventional devices 10, where the suture 11 is a suture and not a sensor probe, the suture 11 is trimmed at or near the surface of the skin once the plug 14 is locked in place. In the embodiment of FIGS. 1 and 2, however, the suture, or sensor probe, 11 can be connected to a monitor (not shown) for detecting bleeding.

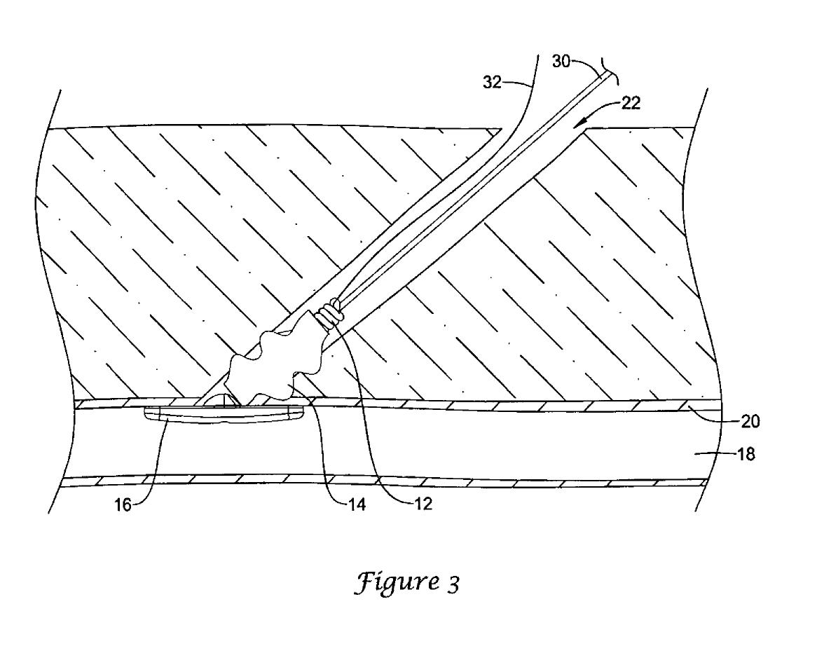

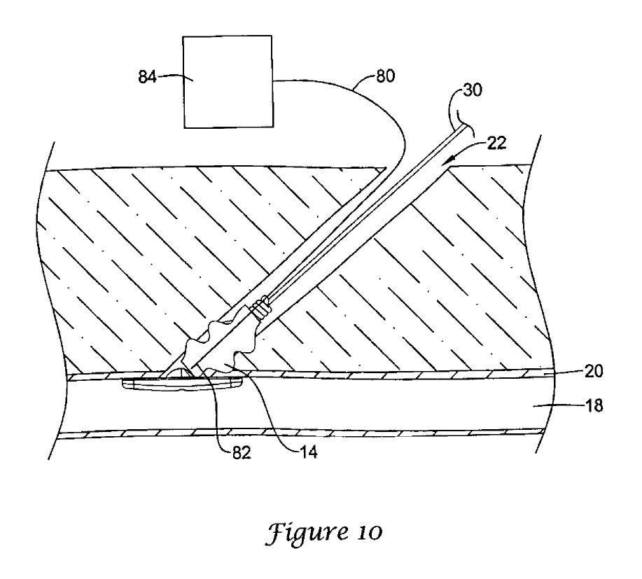

FIG. 3 illustrates another vascular closure device 30 similar to sealing device 10 shown in FIGS. 1 and 2. In this embodiment, the sensor probe 32 is a separate component that is attached to the sealing device 30. In FIG. 3, the sensor probe 32 is attached at its distal end to locking element 12. However, the sensor probe 32 may be attached as desired to other components of the vascular closure device 30. For example, FIG. 10 illustrates another sealing device 30 where a sensor probe 80 is attached at its distal end 82 to a plug.

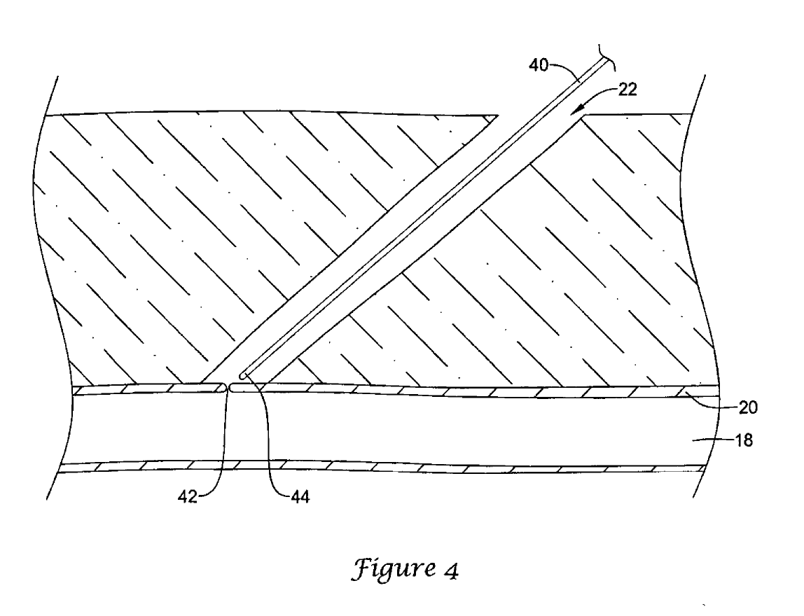

FIG. 4 illustrates an embodiment where a sensor probe 40 is a separate component that is not integrated with or attached to a sealing or vascular closure device. Sensor probe 40 is shown in wound 22 with distal end 44 proximate puncture site 42 in the wall 20 of the blood vessel 18. Such an embodiment may be of use where manual compression is used to seal the vascular puncture site. Such an embodiment may also be suitable for use with an off-the-shelf conventional sealing device and may be introduced into the wound 22 once the sealing device has been used.

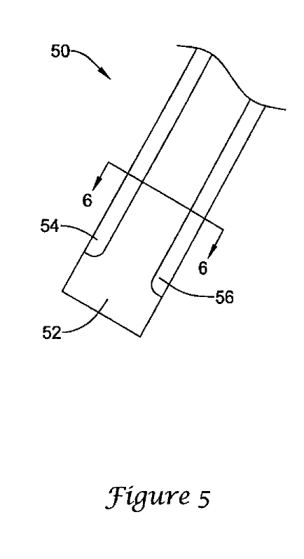

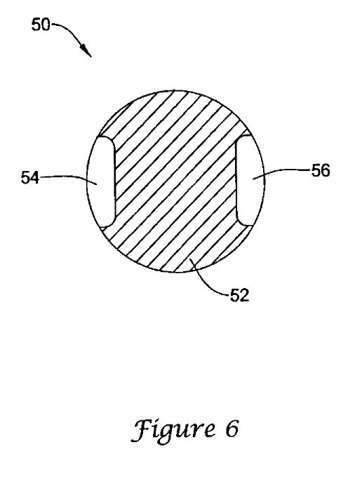

FIGS. 5 and 6 are schematic illustrations of the distal tip of a sensor probe 50 according to an embodiment of the present disclosure, where FIG. 5 is a side view and FIG. 6 is a cross-sectional view. Sensor probe 50 includes a non-electrically conductive elongate member 52 with two spaced apart electrical conductors 54 and 56. The elongate member 52 may be a suture (e.g. device 10 illustrated in FIG. 1, as described herein) or wire, and may have a diameter of about 0.015 inches or less. The two conductors 54 and 56 form a conductive path that extends from a proximal portion (not shown) of the sensor probe 50, down the elongate member 52 and back up the other side of the elongate member 52. The proximal portion of the sensor probe 50 is configured to be attached to a monitor (not shown) where the conductors 54 and 56 are electrically attached to inputs of the monitor. There is a fixed gap of known dimension between the distal ends of conductors 54 and 56. The distal ends of conductors 54 and 56 may terminate short of the distal end of the elongate member 52, as shown, or may, in other embodiments, extend to the distal end of the elongate member 52.

The sensor probe 50 of this embodiment can operate by detecting resistance. In a dry environment, there is a high resistance between the conductors 54 and 56 because there is no conductive path between the conductors 54 and 56 to complete a circuit. In a moist environment such as in a healthy retroperitoneum where no blood has collected but where the body tissue has a normal amount of moisture, there will be a measurable resistance between the conductors 54 and 56. In a wet environment where, for example, blood is actively exiting the arteriotomy, which may indicate an RPH or hemorrhage, the measured resistance will be lower than that measured in the moist but healthy retroperitoneum. The monitor connected to the conductors 54 and 56 can be set to provide a signal when the measured resistance drops below a pre-set level.

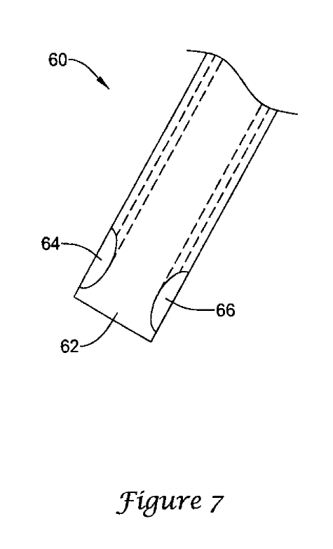

FIG. 7 illustrates a schematic illustration of a sensor probe 60 that includes a non-electrically conductive elongate member 62 and two electrical conductors 64 and 66. This embodiment differs from that of FIG. 6 in that the conductors 64 and 66 are exposed only at their distal ends and where they are connected to the monitor at their proximal ends. From their distal ends to their proximal ends 64 and 66, the conductors are electrically insulated not only from each other but also from the environment. In this manner, the path of a conductive circuit can be more precisely controlled.

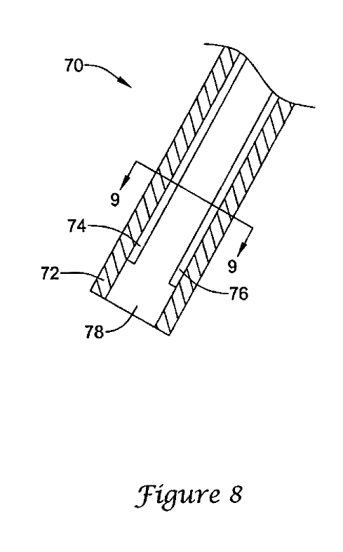

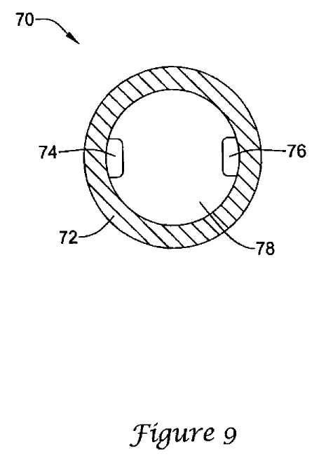

FIGS. 8 and 9 are side and cross-sectional views, respectively, of a sensor probe 70 where the two electrical conductors 74 and 76 are situated within the lumen 78 of a non-electrically conductive elongate member 72. In this embodiment, when the sensor probe 70 is inserted into the wound as described herein, blood would have to enter the distal opening of lumen 78 and reach the conductors 74 and 76 to affect the resistance measurement.

The above embodiments have been described with respect to resistive technology, but the invention is not so limited. For example, a capacitive or optical sensing technology may be used, as explained herein. In a capacitive sensor probe, the capacitor may be open to the environment and the dielectric may change in response to environmental conditions. Blood and other bodily fluids have a lower capacitance than air or most common polymers. Thus a capacitance of a pre-selected level or below may indicate a wet environment, which as discussed above, may signal an RPH or hemorrhage condition. An optical sensor probe may include an input element and a sensor element, and relies on changes in the spectrum received by the sensor element to monitor pooling blood. Both the input element and the sensor element may be fiber optic threads. Light or infrared energy may be transmitted through the input element and received by the output element. The spectrum received by the sensor element is changed based on what the light or infrared energy is reflected against or transmitted through. Upon the detection of an appropriate spectrum, the monitor sends a signal to indicate the presence of blood. Other sensor probes may be configured and used to detect excess water, changes in pH, temperature, blood pressure or blood hematocrit.

FIG. 10 illustrates an embodiment in use. Once the closure device 30 is in place with the sensor probe 80 attached or otherwise disposed in the wound cavity 22, the sensor probe 80 is attached to a monitor 84. The monitor 84 may be configured to display the measured resistance or other datum or may be configured to emit a signal when a pre-set level is reached. The sensor probe 80 may be left in the wound cavity 22 for a period of time following a clinical procedure. The risk of RPH, hemorrhage or other condition drops as time passes. The sensor probe 80 may therefore be left in the wound cavity 22 for a predetermined amount of time and removed once a set time period has passed. For example, the sensor probe 80 may be disconnected from the monitor 84 and removed from the patient's body 90 minutes, 2 hours, 3 hours, 4 hours or 8 hours after the clinical procedure. The sensor probe 80, being a thin wire or suture, may simply be carefully pulled from the wound. Alternatively, the sensor probe 80 may be made from a degradable material that will dissolve after a number of weeks, and the portion of the sensor probe outside the patient's body may be cut away.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A vascular closure device, comprising
an anchor member;
a compressible plug;
a locking mechanism; and
a sensor probe, the sensor probe including a first electrically conductive member having a proximal end and a distal end and a second electrically conductive member having a proximal end and a distal end, the first electrically conductive member being electrically insulated from the second electrically conductive member from the proximal end to the distal end.

2. The vascular closure device of claim 1, wherein the sensor probe comprises a flexible elongate member, wherein the first electrically conductive member extends along a first side of the flexible elongate member and wherein the second electrically conductive member extends parallel to the first electrically conductive member.

3. The vascular closure device of claim 2, wherein the second electrically conductive member extends along a second side of the flexible elongate member, the second side of the flexible elongate member being located opposite the first side.

4. The vascular closure device of claim 1 wherein the sensor probe is unattached to the anchor member, locking mechanism or plug.

5. The vascular closure device of claim 1 wherein the sensor probe has a distal end attached to the locking mechanism.

6. The vascular closure device of claim 1 wherein the sensor probe has a distal end attached to the plug.

7. The vascular closure device of claim 1 wherein the distal ends of the first and second electrically conductive members are proximal a distal end of the sensor probe.

8. The vascular closure device of claim 1 further comprising insulation disposed about the first and second electrically conductive members, said insulation having a distal end that is proximal of the distal ends of the first and second electrically conductive members.

9. The vascular closure device of claim 1 wherein the flexible elongate member is hollow and has an inside surface and wherein the first and second electrically conductive members are disposed on the inside surface.

10. A monitoring system, comprising:
a vascular closure device;
a sensor probe having a distal end attached to the vascular closure device; and
a monitor operatively connected to the sensor probe, the monitor configured to receive a signal from the sensor probe and to output information based on the signal.

11. The system of claim 10, wherein the signal is an electrical resistance.

12. The system of claim 10, wherein the signal is a capacitance.

13. The system of claim 10, wherein the signal is an optical signal.

14. The system of claim 10 wherein the monitor is configured to deliver an audible or visible communication when the signal reaches a pre-determined level.

15. The system of claim 10, wherein the sensor probe includes a first electrically conductive member having a proximal end and a distal end and a second electrically conductive member having a proximal end and a distal end, the first electrically conductive member being electrically insulated from the second electrically conductive member from the proximal end to the distal end.

16. The vascular closure device of claim 15, wherein the sensor probe comprises a flexible elongate member, wherein the first electrically conductive member extends along a first side of the flexible elongate member and wherein the second electrically conductive member extends parallel to the first electrically conductive member, and wherein the second electrically conductive member extends along a second side of the flexible elongate member, the second side of the flexible elongate member being located opposite the first side.

17. The vascular closure device of claim 15 wherein the sensor probe is a suture of the vascular closure device.

18. The vascular closure device of claim 15 wherein the distal ends of the first and second electrically conductive members are proximal a distal end of the sensor probe.

19. The vascular closure device of claim 15 further comprising insulation disposed about the first and second electrically conductive members, said insulation having a distal end that is proximal of the distal ends of the first and second electrically conductive members.

20. The vascular closure device of claim 15 wherein the flexible elongate member is hollow and has an inside surface and wherein the first and second electrically conductive members are disposed on the inside surface.
